(19) United States
(12) Patent Application Publication
(10) Pub. No.: US 2008/0081027 A1
(43) Pub. Date: Apr. 3, 2008

(54) Concealer composition

(75) Inventor: Kojo Tanaka, Ashiya (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/904,528

(22) Filed: Sep. 27, 2007

Related U.S. Application Data
(60) Provisional application No. 60/847,781, filed on Sep. 28, 2006.

(51) Int. Cl. A61K 8/29; A61Q 1/02

(52) U.S. Cl. 424/63

(57) ABSTRACT

Disclosed is a concealer composition comprising by weight from about 30% to about 99% of powders, the powders comprising by weight of the entire composition; (a) from about 0.1% to about 74.9% of a titanium coated pigment made of a core pigment and a titanium dioxide coating, the amount of titanium dioxide coating accounting for at least 33% of the titanium coated pigment; and (b) from about 0.1% to about 98.9% of a metal soap coated pigment made of a core pigment and a metal soap coating, the amount of metal soap coating accounting for at least 1% of the metal soap coated pigment; wherein the composition comprises at least 25% of a high coverage pigment selected from the group consisting of titanium dioxide, zinc oxide, and mixtures thereof, wherein the amount of high coverage pigment includes the titanium dioxide coating of the titanium coated pigment; and wherein when the high coverage pigment is used as the core pigment of the metal soap coated pigment, such amount of high coverage pigment is also included.

### CROSS REFERENCE TO RELATED APPLICATIONS

This application claims the benefit of U.S. Provisional Application No. 60/847,781 filed on Sep. 28, 2006.

### FIELD OF THE INVENTION

The present invention relates to a concealer composition which provides an improved natural coverage to skin imperfections. The present invention further relates to methods of make-up using such composition.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Concealers are used to obtain high coverage on a particularly concerned area, and supplement the function of a foundation on. Concealers are typically liquid, paste or semi-solid form products containing a high level of pigments having opacity, such as titanium dioxide, and are typically used prior to applying the foundation.

One of the unsatisfying points for concealer users is that, the portion of skin for which the concealer is applied provides an unnatural appearance. Namely, in view of different texture or different color provided by the concealer, the skin area on which concealer is applied is noticeable. Considering that concealers are used in desire to make a certain concerned area of the skin to be less conspicuous from the surrounding area, such unnatural appearance is contrary to what is desired.

Another unsatisfying point for concealer users is that, concealer products are typically in liquid form and are designed to apply to the skin prior to application of foundation. Thus, concealer products are inconvenient to carry, and inconvenient to use as touch-up during the day.

Concealers are disclosed in, for example, Japanese Patent Publications 2000-327532, 2004-008307, 2005-298482, 6-56628, and 2003-277217. There are no references, however, which disclose concealers that provide satisfactory natural coverage and good adhesion at the same time.

Based on the foregoing, there is a need for a concealer which provides improved natural coverage to skin imperfections, and also has good adhesion to the skin. None of the existing art provides all of the advantages and benefits of the present invention.

### SUMMARY

The present invention is directed to a concealer composition comprising by weight from about 30% to about 99% of powders, the powders comprising by weight of the entire composition:

(a) from about 0.1% to about 74.9% of a titanium coated pigment made of a core pigment and a titanium dioxide coating, the amount of titanium dioxide coating accounting for at least 33% of the titanium coated pigment; and
(b) from about 0.1% to about 98.9% of a metal soap coated pigment made of a core pigment and a metal soap coating, the amount of metal soap coating accounting for at least 1% of the metal soap coated pigment;
wherein the composition comprises at least 25% of a high coverage pigment selected from the group consisting of titanium dioxide, zinc oxide, and mixtures thereof,
wherein the amount of high coverage pigment includes the titanium dioxide coating of the titanium coated pigment; and
wherein when the high coverage pigment is used as the core pigment of the metal soap coated pigment, such amount of high coverage pigment is also included.

The present invention is also directed to a method of making up the skin utilizing the aforementioned cosmetic product.

These and other features, aspects, and advantages of the present invention will become evident to those skilled in the art from a reading of the present disclosure with the appended claims.

### DETAILED DESCRIPTION OF THE INVENTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Concealer and Method of Use

The present composition is a concealer comprising at least about 25% by weight of the concealer of high coverage pigment. Herein, a concealer is a composition which provides higher coverage than a foundation, and is typically used to a particular area of the skin for which specific care of appearance is desired. The high amount of high coverage pigment provides such high coverage.

The present concealer comprises from about 30% to about 99% of powder, including the specific pigments described hereinbelow. The present composition may take a variety of product forms, including loose powder, pressed powder, semi-solid, paste, and liquid. Preferably, the present concealer is used with a foundation of the same product form. Without being bound by theory, it is believed that, by matching the product form of the foundation and concealer, the texture of the two compositions, when applied to the skin, are very similar, such that the concealer provides natural coverage to the portion of the skin where higher coverage is needed. Preferably, the present composition comprises from about 75% to about 99% of powder, and is in powder form.

Preferably, the present composition has a color by comprising colored powders as described hereinbelow, dyes, and other colorant materials. More preferably, the CMC DE of the concealer color is less than 1.5 compared to the foundation color with which the concealer is used. Accordingly, there is substantially no difference in color of the foundation and concealer in the eyes of the general consumer. The matching of the color of the foundation and concealer may be provided by 1) indication of color code on independently packaged foundation and concealer, 2) providing the foundation and concealer as a kit, or 3) providing the foundation and concealer in the same package.

In one preferred embodiment, the foundation and concealer have substantially no difference in color, and are of the same product form. By having such common color and product form, the combined use of the foundation and concealer provides a seamless appearance to the skin, namely, the portion where concealer were applied is not recognizable.

In one preferred embodiment, the foundation and concealer are both powder form, and are provided in the same package. The package is a compact housing a pan for the foundation, a pan for the concealer, and an applicator. Preferably, two applicators are contained in the package such that the foundation and concealer can be used via separate applicators. Suitable for foundation is a sponge for broad application, and suitable for concealer is a tip for focused application.

The present invention relates to the method of making up the skin using the present concealer. Conventionally, concealers are recommended for use prior to application of the foundation. The present concealer may be used either prior to or after application of the foundation. For providing the concealer suitable for “after application” the concealer is preferably substantially the same color as the foundation. By providing such color the present concealer can be used as a touch up during the day.

### Titanium Coated Pigment

The present concealer composition comprises from about 0.1% to about 74.9%, preferably from about 1% to about 60%, of a titanium coated pigment, the titanium dioxide coating accounting for at least 33% of the titanium coated pigment, preferably at least 40% of the titanium coated pigment. The titanium coated pigment herein is made of a core pigment selected from the group consisting of talc, mica, sericite, synthetic mica, aluminum oxide, silica, boron nitride, and mixtures thereof, which core pigment is coated with titanium dioxide having a particle size of from about 100 nm to about 500 nm.

The present titanium coated pigment has a coating of titanium dioxide higher than those widely used in the art. By such higher coating, the present titanium coated pigments provide a high coverage effect compared to the same weight of pigments made solely of titanium dioxide.

Commercially available titanium coated pigments highly useful herein include 57.3% titanium dioxide coated sericite further coated with methicone in the name of Fancyveil S-3060SW available from Shokubai Kasei.

### Metal Soap Coated Pigment

The present concealer composition comprises from about 0.1% to about 98.9%, preferably from about 1% to about 70%, of a metal soap coated pigment, the metal soap coating accounting for at least 1% of the metal soap coated pigment, preferably at least 3% of the metal soap coated pigment. The metal soap coated pigment herein is made of a core pigment selected from the group consisting of sericite, talc, mica, synthetic mica, titanium dioxide, aluminum oxide, silica, boron nitride, and mixtures thereof, which core pigment is coated with one or more metal soap, such as aluminum dimyristate, aluminium stearate, magnesium stearate, zinc myristate, magnesium myristate, zinc palmitate, zinc laurate, calcium stearate, and mixtures thereof.

The present metal soap coated pigment has a coating of metal soap higher than those widely used in the art. By such higher coating, the present metal soap coated pigment provides good adhesion between itself and the skin, as well as between the pigments themselves.

Commercially available metal soap coated pigments highly useful herein include 5% aluminum dimyristate coated mica with the tradenames 5MI SERICITE and 5MI MICA M-102, 5% aluminum dimyristate coated talc with the tradenames 5MI TALC JA-46R, titanium dioxide coated with 5% aluminum dimyristate with the tradename 5MI Titanium Dioxide, all available from Miyoshi Kasei.

### High Coverage Pigment

The present concealer composition at least about 25% by weight of high coverage pigment, including the amount of titanium dioxide coating of the titanium coated pigment described hereinabove. Further, when the high coverage pigment is used as the core pigment of the metal soap coated pigment, such amount of the high coverage pigment as core pigment is also included.

Preferably, the amount of high coverage pigment is no more than about 60%, more preferably no more than about 50%.

The high coverage pigment herein is selected from the group consisting of titanium dioxide, zinc oxide, and mixtures thereof, and have an average particle size of from about 100 nm to about 500 nm, preferably from about 200 nm to about 350 nm. The titanium dioxide may be rutile type or anatase type. The high coverage pigment can be used as is, but preferably at least partially used as complex pigments with other organic/inorganic and are also preferably hydrophobically coated. The titanium coated pigment described hereinabove is a highly referred complex pigment.

As mentioned above, the titanium coated pigments have a high coverage effect compared to the same weight of pigments made solely of titanium dioxide. By incorporating the metal soap coated pigments, the titanium coated pigments as well as other pigments have good adhesion to the skin and with each other, such that a thicker layer of concealer can be applied to the skin. Without being bound by theory, by combining the titanium coated pigment and the metal soap coated pigment, it is believed that a concealer having high coverage is obtained for total amount of titanium dioxide contained in the composition. There is interest to control the total amount of titanium dioxide and overall high coverage pigment in the formulation at a low level, as too much high coverage pigment in the formulation may provide a different texture from a foundation, and thus provide an unnatural look.

Commercially available high coverage pigments highly useful herein include those mentioned above as titanium coated pigments, and Titanium Dioxide coated with Methicone with tradename SI Titanium Dioxide IS available from Miyoshi Kasei.

### Other Powders

The present concealer composition may contain powders other than those above for color, and other appearance and skin feel effects. For providing products of powder form, the total powder content is at least about 75% of the foundation composition or the concealer composition.

In the present invention, the powders are selected such that the color of the concealer is substantially the same as the foundation with which it is to be used. Powders other than those described herein above may be used.

The other powders useful herein include those that provide color or change tone, and also those that provide a certain skin feel. Useful powders herein include clay mineral powders such as talc, mica, sericite, bentonite and montomorilonite; coloring powders useful herein include iron oxides, iron titate, ultramarine blue, Prussian blue, chromium oxide, chromium hydroxide, cobalt oxide, cobalt titanate, titanium oxide coated mica, laked tar color dyes, and laked natural color dyes; organic powders herein include polyacrylates such as methyl methacrylate copolymer and methyl methacrylate crosspolymer, celluloses, polyalkylenes such as polyethylene and polypropylene, vinyl acetates, polystyrenes such as styrene-acrylic acid copolymers, polyamides such as 12-nylon and 6-nylon, acrylic acid ethers such as acrylic acid methyl ether and acrylic acid ethyl ether, polyvinyl pyrrolidones; vinyl chloride polymers, silicones such as polyorganosilsesquioxane resin and solid silicone elastomers, tetrafluoroethylene polymer, and fish scale guanine; inorganic powders such as barium sulfate, calcium secondary phosphate, hydroxy apatite, silicates; such as calcium silicate, magnesium silicate, barium silicate and aluminium silicate, silica beads, metal dioxides such as zirconium oxide and aluminium hydroxide, carbonates such as calcium carbonate and magnesium carbonate, boron nitride and synthetic fluorphlogopite.

In one preferred embodiment, polyorganosilsesquioxane resin and solid silicone elastomers may be used for enhancing the effect of hiding skin pores.

In another preferred embodiment, mica may be conveniently used for powder form compositions, for prevention of caking and providing appropriate pay off upon application. The powders herein may be surface coated with a coating material having hydrophobic characteristics, or lipophobic hydrophobic characteristics. Useful hydrophobic coating materials herein include methyl polysiloxane, methyl hydrogen polysiloxane, methyl phenyl polysilxoane, n-octyl triethoxy silane, methyl-alpha-styrene polysiloxane, acryl silicone copolymer, and mixtures thereof. Useful lipophobic hydrophobic coating materials are fluorine compounds such as perfluorooctyl triethoxylsilane, perfluoroalkylphosphoric acids, their salts, and mixtures thereof.

Commercially available powders highly useful herein include methyl methacylate copolymer with tradename GANZ PEARL series available from Ganz Chemical Co., Ltd., and SYLYSIA series available from Fuji Sylysia Chemical, Nylon-12 with tradename NYLON POWDER series available from Toray Dow Corning, vinyl dimethicone/methicone silsesquioxane crosspolymer with tradenames KSP series available from ShinEtsu Chemical Co., Ltd., Tokyo Japan, hardened polyorgano siloxane elastomers with tradenames TREFIL series available from Toray Dow Corning, boron nitride with tradename SHP series available from Mizushima Ferroalloy Co., Ltd.

### Carrier and Product Form

The present concealer composition further contains an appropriate carrier for providing the desired product form. Such carrier may include non-volatile oil, lipophilic surfactant, humectant, water, volatile silicone oil, and thickeners such as fatty compound, solid wax, gelling agent, inorganic thickening agent, and silicone elastomer. For providing a powder form, the powders are mixed with a binder comprising one or more of non-volatile oil, lipophilic surfactant, and humectant. In one preferred embodiment, the composition comprises: by weight from about 75% to about 99% of powders, the powders comprising by weight of the entire composition:

(a) from about 1% to about 60% of the titanium coated pigment, the titanium coating accounting for at least 40% of the titanium coated pigment;
(b) from about 1% to about 70% of the metal soap coated pigment, the metal soap coating accounting for at least 3% of the metal soap coated pigment; and
by weight from about 1% to about 25% of a binder selected from the group consisting of non-volatile oil, lipophilic surfactant, humectant, and mixtures thereof;
wherein the composition comprises from about 25% to about 60% of the high coverage pigment.

For providing an emulsified composition, non-volatile oil, liphophilic surfactant, water, and optional surfactant and thickeners may be used. Thickeners may be used to control viscosity or solidify the composition. Volatile silicone oils may be used for providing freshness to the skin, and favorably used in liquid product forms.

### Non-Volatile Oil

Useful for the composition of the present invention is a non-volatile oil. The non-volatile oil herein is believed to provide improved smoothness to the skin, and also alleviate dry feeling of the skin. Non-volatile oils can also be used as a binder for powder forms of the present composition.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexylan available from Nisshin Seiyu, and tocopherol acetates available from Eisai.

Non-volatile oils useful herein also include polyalkyl or polyaryl siloxanes with the following structure (I)

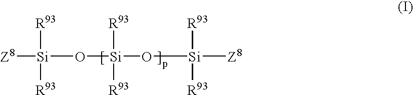

wherein R93 is alkyl or aryl, and p is an integer from about 7 to about 8,000. Z8 represents groups which block the ends of the silicone chains. The alkyl or aryl groups substituted on the siloxane chain (R93) or at the ends of the siloxane chains Z8 can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z8 groups include hydroxy, methyl, methoxy, ethoxy, propoxy, and aryloxy. The two R93 groups on the silicon atom may represent the same group or different groups. Preferably, the two R93 groups represent the same group. Suitable R93 groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

### Lipophilic Surfactant

Useful for the composition of the present invention is a lipophilic surfactant. When incorporated in water-in-oil emulsions, the amount included is preferably from about 0.1% to about 25%. When incorporated in solid water-in-oil emulsion forms, the amount included is preferably from about 1% to about 20%. Without being bound by theory, the species and levels of the lipophilic surfactant herein are believed to provide a stable water-in-oil emulsion in view of the other components of the present invention. Lipophilic surfactants can also be used as a binder for powder forms of the present composition. The lipophilic surfactant herein has an HLB value of less than about 8.

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), (iii), and (iv) as shown below, and mixtures thereof.

(i) dimethicone copolyols having the formulation:

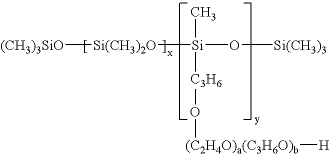

wherein x is an integer from 5 to 100, y is an integer from 1 to 50, a is zero or greater, b is zero or greater, the average sum of a+b being 1-100.
(ii) dimethicone copolyols having the formulation:

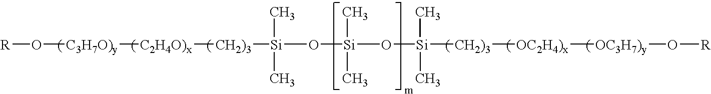

wherein R is selected from the group consisting of hydrogen, methyl, and combinations thereof, m is an integer from 5 to 100, x is independently zero or greater, y is independently zero or greater, the sum of x+y being 1-100.
(iii) branched polyether-polydiorganosiloxane emulsifiers herein having the formulation:

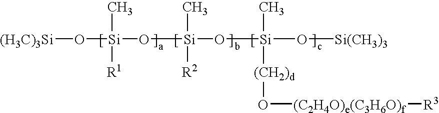

wherein R1 is an alkyl group having from about 1 to about 20 carbons; R2 is

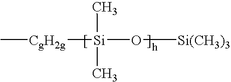

wherein g is from about 1 to about 5, and h is from about 5 to about 20; R3 is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.
(iv) alkyl dimethicone copolyols which are nonionic polysiloxane copolymer having emulsifying ability, comprising a methylpolysiloxane moiety, an alkyl methylpolysiloxane moiety, and a poly(oxyalkylene)methylpolysiloxane moiety; having an HLB from about 4 to about 6, and a molecular weight of from about 10,000 to about 20,000, wherein the alkyl group is made of from about 10 to about 22 carbons. Suitable alkyl dimethicone copolyols herein are those which have the following formulation:

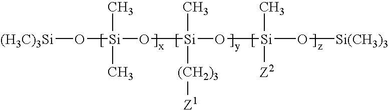

wherein Z1 is O(C2H4O)p(C3H6O)qH, p is from 0 to about 50, q is from 0 to about 30, wherein p and q are not 0 at the same time; x is from 1 to about 200, y is from 1 to about 40, and z is from 1 to about 100, and Z2 is an alkyl group having from about 10 to about 22 carbons, preferably from about 16 to about 18 carbons.

Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Corning, and branched polyether-polydiorganosiloxane emulsifiers such as PEG-9 polydimethylsiloxyethyl Dimethicone, having an HLB of about 4 and a molecular weight of about 6,000 having a tradename KF 6028 available from ShinEtsu Chemical. Highly preferred alkyl dimethicone copolyols include cetyl dimethicone copolyol and stearyl dimethicone copolyol. A highly preferred commercially available alkyl dimethicone copolyol includes cetyl dimethicone copolyol, also called Methylpolysiloxane Cetylmethylpolysiloxane Poly(oxyethylene oxypropylene) Methylpolysiloxane Copolymer, having an HLB of about 5 and a molecular weight of about 13,000 having a tradename ABIL EM90 available from Goldschmidt Personal Care.

### Humectant

Useful for the composition of the present invention is a humectant. Humectants can be used for emulsions as well as a binder for powder forms of the present composition.

The humectants herein are selected from the group consisting of polyhydric alcohols, water soluble alkoxylated nonionic polymers, and mixtures thereof. Polyhydric alcohols useful herein include glycerin, propylene glycol, 1,3-butylene glycol, dipropylene glycol, diglycerin, sodium hyaluronate, and mixtures thereof.

Commercially available humectants herein include: glycerin available from Asahi Denka; propylene glycol with tradename LEXOL PG-865/855 available from Inolex, 1,2-PROPYLENE GLYCOL USP available from BASF; 1,3-butylene glycol available from Kyowa Hakko Kogyo; dipropylene glycol with the same tradename available from BASF; diglycerin with tradename DIGLYCEROL available from Solvay GmbH; sodium hyaluronate with tradenames ACTIMOIST available from Active Organics, AVIAN SODIUM HYALURONATE series available from Intergen, HYALURONIC ACID Na available from Ichimaru Pharcos.

### Water

The water-in-oil emulsion compositions of the present invention comprise water in an amount sufficient to provide a discontinuous aqueous phase. In the present invention, deionized water is typically used. Water from fermented biological cultures or its filtrates, and from natural sources including mineral cations can also be used, depending on the desired characteristic of the product. A highly preferred water source of this kind is Saccharomycopsis Ferment Filtrate with tradename SK-II Pitera available from Kashiwayama.

### Volatile Silicone Oil

Volatile silicone oils may be used for the present invention. The volatile silicone oil useful herein are selected from those having a boiling point of from about 60 to about 260° C., preferably those having from 2 to 7 silicon atoms.

The volatile silicone oils useful herein include polyalkyl or polyaryl siloxanes with the following structure (I):

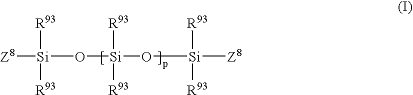

wherein R93 is independently alkyl or aryl, and p is an integer from about 0 to about 5. Z8 represents groups which block the ends of the silicone chains. Preferably, R93 groups include methyl, ethyl, propyl, phenyl, methylphenyl and phenylmethyl, Z8 groups include hydroxy, methyl, methoxy, ethoxy, propoxy, and aryloxy. More preferably, R93 groups and Z8 groups are methyl groups. The preferred volatile silicone compounds are hexamethyldisiloxane, octamethyltrisiloxane, decamethyltetrasiloxane, hexadecamethylheptasiloxane. Commercially available volatile silicone compounds useful herein include octamethyltrisiloxane with tradename SH200C-1cs, decamethyltetrasiloxane with tradename SH200C-1.5cs, hexadecamethylheptasiloxane with tradename SH200C-2cs, all available from Dow Corning.

The volatile silicone oils useful herein also include a cyclic silicone compound having the formula:

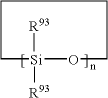

wherein R93 is independently alkyl or aryl, and n is an integer of from 3 to 7.

Preferably, R93 groups include methyl, ethyl, propyl, phenyl, methylphenyl and phenylmethyl. More preferably, R93 groups are methyl groups. The preferred volatile silicone compounds are octamethylcyclotetrasiloxane, decamethylcyclopentasiloxane, tetradecamethylcyclohexasiloxane. Commercially available volatile silicone compounds useful herein include octamethylcyclotetrasiloxane with tradename SH244, decamethylcyclopentasiloxane with tradename DC245 and SH245, and dodeamethylcyclohexasiloxane with tradename DC246; all available from Dow Corning.

### Thickener

Thickeners may be used for the present invention. Thickeners can be used for adding viscosity to liquid forms, and for solidifying compositions. When used in liquid forms, the thickener is kept to about 5% of the entire composition. Solid water-in-oil emulsion forms comprise thickeners, typically solid wax, for solidifying the composition.

The thickeners useful herein are selected from the group consisting of fatty compounds, solid wax, gelling agents, inorganic thickeners, silicone elastomers, and mixtures thereof. The amount and type of thickeners are selected according to the desired viscosity and characteristics of the product.

### Fatty Compounds

Fatty compounds useful herein include stearic acid, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

### Solid Wax

The solid waxes useful herein are paraffin wax, microcrystalline wax, ozokerite wax, ceresin wax, carnauba wax, candellila wax, eicosanyl behenate, and mixtures thereof. A mixture of waxes is preferably used.

Commercially available solid waxes useful herein include: Candelilla wax NC-1630 available from Cerarica Noda, Ozokerite wax SP-1021 available from Strahl & Pitsh, and Eicosanyl behenate available from Cas Chemical.

### Gelling Agents

The gelling agents useful as thickeners of the present invention include esters and amides of fatty acid gellants, hydroxy acids, hydroxy fatty acids, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:

R2-NH—CO—(CH2)2-CH—(NH—CO—R1)-CO—NH—R2

wherein R1 is an aliphatic hydrocarbon radical having from about 12 to about 22 carbon atoms, and R2 is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercially available with tradename Gelling agent GP-1 available from Ajinomoto.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:

R1-CO—(CH2)10-CH—(OH)—(CH2)5-CH3

wherein R1 is R2 or NR2R3; and R2 and R3 are hydrogen, or an alkyl, aryl, or arylalkyl radical which is branched linear or cyclic and has from about 1 to about 22 carbon atoms; preferably, from about 1 to about 18 carbon atoms. R2 and R3 may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1-propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

### Inorganic Thickeners

Inorganic thickeners useful herein include hectorite, bentonite, montmorillonite, and bentone clays which have been modified to be compatible with oil. Preferably, the modification is quaternization with an ammonium compound. Preferable inorganic thickeners include quaternary ammonium modified hectorite. Commercially available oil swelling clay materials include benzyldimethyl stearyl ammonium hectorite with tradename Bentone 38 available from Elementis.

### Silicone Elastomers

Suitable for use herein are silicone elastomers which can be emulsifying or non-emulsifying crosslinked siloxane elastomers or mixtures thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Non-emulsifying elastomers useful in the present invention are formed via crosslinking organohydroenpolysiloxanes with an alpha, omega-diene. Emulsifying elastomers herein include polyoxyalkylene modified elastomers formed via crosslinking from organohydrogenpolysiloxanes with polyoxyalkylene dienes or organohydrogenpolysiloxanes containing at least one polyether group crosslinked with an alpha, omega-diene. Emulsifying crosslinked organopolysiloxane elastomer can notably be chosen from the crosslinked polymers described in U.S. Pat. Nos. 5,412,004, 5,837,793, and 5,811,487. In addition, an emulsifying elastomer comprised of dimethicone copolyol crosspolymer (and dimethicone) is available from Shin Etsu under the tradename KSG-21.

Non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. Nos. 4,970,252, 5,760,116, and 5,654,362. Additional crosslinked organopolysiloxane elastomers useful in the present invention are disclosed in Japanese Patent Application JP 61-18708, assigned to Pola Kasei Kogyo K K. Commercially available elastomers preferred for use herein are Dow Corning's 9040 silicone elastomer blend, Shin Etsu's KSG-21, and mixtures thereof.

### Additional Components

The composition of the present invention may include other additional components, which may be selected by the artisan according to the desired characteristics of the final product and which are suitable for rendering the composition more cosmetically or aesthetically acceptable or to provide them with additional usage benefits.

Other components which can be formulated into the compositions of the present invention are; preservatives such as benzyl alcohol, methyl paraben, propyl paraben, sodium dehydro acetate, niacinamide, imidazolidinyl area, and EDTA and its salts, perfumes, ultraviolet and infrared screening and absorbing agents, and others.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Composition for Examples 1-4

| | | Ex. 1 | Ex. 2 | Ex. 3 | Ex. 4 |
|---|---|---|---|---|---|
| 1 | Talc coated with 5% Aluminum Dimyristate *1 | 5.74 | | | 3.32 |
| 2 | Sericite coated with 5% Aluminum Dimyristate *2 | 23 | | | |
| 3 | Mica coated with 5% Aluminum Dimyristate *3 | 17.5 | 29.05 | | |
| 4 | Sericite coated with 57.3% Titanium Dioxide and | 5 | 50 | 10 | 20 |
| | Methicone *4 | | | | |
| 5 | Titanium Dioxide coated with 5% Aluminum | | | 25 | 18 |
| | Dimyristate *5 | | | | |
| 6 | Titanium Dioxide coated with Methicone *6 | 27 | | | |
| 7 | Polymethyl methacrylate *7 | 8 | | | |
| 8 | Vinyl Dimethicone/Methicone Silesquioxane | | | 1.8 | |
| | Crosspolymer *8 | | | | |
| 9 | Mica coated with Titanium Dioxide *9 | | | | 0.1 |
| 10 | Yellow Iron Oxide coated with Methicone *10 | 3.84 | 5.5 | 2.7 | 3 |
| 11 | Black Iron Oxide coated with Methicone *11 | 0.4 | 0.48 | 0.2 | 0.25 |
| 12 | Red Iron Oxide coated with Methicone *12 | 0.82 | 1.17 | 0.3 | 0.33 |
| 13 | Sorbitan Monoisostearate *13 | | | 3 | |
| 14 | Dimethicone Copolyol *14 | | | 15 | |
| 15 | Dimethicone *15 | 3.85 | 8.85 | | |
| 16 | Cyclopentasiloxane *16 | | | 12 | |
| 17 | Neopentyl Glycol Dioctanoate *17 | | | | 24.35 |
| 18 | Hydrogenated Castor Oil *18 | | | | 10 |
| 19 | Ethylhexyl Methoxycinnamate *19 | 4 | 4 | | |
| 20 | D-delta-tocopherol *20 | 0.1 | 0.1 | | 0.1 |
| 21 | Lanolin *21 | | | | 10 |
| 22 | Ozokerite Wax *22 | 2 | | | |
| 23 | Candelilla Wax *23 | | | | 0.5 |
| 24 | Microcrystalline Wax *24 | | | | 2 |
| 25 | Polyethylene Wax *25 | | | | 5.5 |
| 26 | WATER | | | 19.1 | |
| 27 | Glycerin *26 | | 0.1 | | |
| 28 | Butylene Glycol *27 | | | 5 | |
| 29 | Mulberry Root Extract *28 | | | 0.1 | |
| 30 | Saccharomycopsis Ferment Filtrate *29 | | | 5.0 | |
| 31 | Niacinamide *30 | 0.1 | 0.1 | 0.1 | |
| 32 | Panthenol *31 | 0.1 | 0.1 | 0.1 | |
| 33 | EDTA-2NA | | | 0.1 | |
| 34 | Preservatives | 0.5 | 0.5 | 0.5 | 0.5 |
| 35 | Perfume | 0.05 | 0.05 | | 0.05 |
| | Total | 100 | 100 | 100 | 100 |

Definitions of Components
*1 Talc coated with 5% Aluminum Dimyristate: 5M1-TALC JA-46R available from Miyoshi Kasei
*2 Sericite coated with 5% Aluminum Dimyristate: 5MI-SERICITE available from Miyoshi Kasei
*3 Mica coated with 5% Aluminum Dimyristate: 5M1-MICA M-102 available from Miyoshi Kasei
*4 Sericite coated with 57.3% Titanium Dioxide and Methicone: Fancyveil S-3060SW available from Shokubai Kasei
*5 Titanium Dioxide coated with 5% Aluminum Dimyristate: 5M1-Titanium Dioxide available from Miyoshi Kasei
*6 Titanium Dioxide coated with methicone: SI Titanium Dioxide IS available from Miyoshi Kasei
*7 Polymethyl Methacrylate: GANZ PEARL GM-0600 available from GANZ CHEMICAL CO., LTD
*8 Vinyl Dimethicone/Methicone Silesquioxane Crosspolymer: KSP-100 available from Shinetsu Silicone
*9 Mica coated with Titanium Dioxide: FLAMENCO SUPER PEARL available from THE MEARL
*10 Yellow Iron Oxide coated with Methicone: SI MAPICO YW LIGHT LEMON XLO available from Daito Kasei
*11 Black Iron Oxide coated with Methicone: SI BLACK IRON OXIDE NO.247 available from Daito Kasei
*12 Red Iron Oxide coated with Methicone: SI PURE RED IRON OXIDE R-3098 available from Daito Kasei
*13 Sorbitan Monoisostearate: Crill 6 available from Croda
*14 Dimethicone Copolyol: DC5225C available from Dow Coming
*15 Dimethicone: SILICONE OIL SH200C-50CS available from Dow Corning
*16 Cyclopentasiloxane: 5H245 available from Dow Coming
*17 Neopentyl Glycol Dioctanoate: Cosmol 525 available from Nisshin Oillio
*18 Hydrogenated Castor Oil: Hydrogenated Castor Oil available from Matsumoto Kosho
*19 Ethylhexyl Methoxycinnamate: PARSOL MCX available from ROCHE VITAMINS JAPAN K.K
*20 D-delta-tocopherol: D-DELTA-TOCOPHEROL available from EISAI CO., LTD.
*21 Lanolin: Crodaran SWL available from Croda Japan
*22 Ozokerite Wax: Ozokerite Wax SP-1021 available from Strahl & Pitsh
*23 Candelilla Wax: Candelilla Wax NC-1630 available from Cerarica Noda Co., Ltd
*24 Microcrystalline Wax: MULTIWAX 180-M Yellow available from Witco
*25 Polyethylene Wax: PERFORMALENE PL available from New Phase Technology
*26 Glycerin: Glycerin USP available from Asahi Denka
*27 Butylene Glycol: 1,3 Butylene Glycol available from Kyowa Hakko Kogyo
*28 Mulberry Root Extract: Mulberry BG, available from Maruzen Pharmaceuticals
*29 Saccharomycopsis Ferment Filtrate: SK-II Pitera available from Kashiwayama
*30 Niacinamide: Niacinamide available from Reilly Industries Inc
*31 Panthenol: DL-Panthenol available from Alps Pharmaceutical Ind

### Method of Preparation

The cosmetic compositions of Examples 1 and 2 are prepared as follows:

Component numbers 1-12 and 31-34 are mixed with a mixer to make a pigment component. Separately, component numbers 15-21 and 35 are mixed to make a binder component. The binder component and component numbers 26-30 is added into the pigment component and mixed by a mixer. The obtained composition is pressed in a pan.

The cosmetic composition of Example 3 is prepared as follows:

Component numbers 13-21 are mixed with suitable mixer until homogeneous to make a lipophilic mixture. Component numbers 1-12 are mixed with suitable mixer until homogeneous to make a powder mixture. The powder mixture is pulverized using pulverizer. The powder mixture is added into the lipophilic mixture with suitable mixer until homogeneous. Component numbers 26-34 are dissolved with suitable mixer until all components are completely dissolved to make a water phase. The water phase is added into the product of powder added lipophilic mixture to effect emulsion at room temperature using homogenizer.

The cosmetic composition of Example 4 is prepared as follows:

First, component numbers 1-12 are mixed with a mixer to make a pigment component. Separately, component numbers 13-25, 34, and 35 are mixed and heated using a Propeller Mixer at 95° C. in a sealed tank. After all components are melted, the mixture is cooled to 85° C. After that, the pigment component are added to the tank, and the mixture is dispersed at 85° C. Finally, the dispersion is filled in an air-tight container and allowed to cool to room temperature.

### Usage

These embodiments represented by the previous examples have many advantages when applied to the skin as make up compositions. Examples 1 and 2 are useful as powder concealer products. Example 3 is useful as a liquid concealer product. Example 4 is useful as a stick concealer product. The products of Examples 1-4, when applied to the skin, provide natural coverage and good adhesion to the skin. Further, when the products of Examples 1-4 are used with a foundation that has the same product form and has substantially the same color, respectively, the combined usage of the foundation and concealer provides a seamless appearance.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A concealer composition comprising by weight from about 30% to about 99% of powders, the powders comprising by weight of the entire composition:
(a) from about 0.1% to about 74.9% of a titanium coated pigment made of a core pigment and a titanium dioxide coating, the amount of titanium dioxide coating accounting for at least 33% of the titanium coated pigment; and
(b) from about 0.1% to about 98.9% of a metal soap coated pigment made of a core pigment and a metal soap coating, the amount of metal soap coating accounting for at least 1% of the metal soap coated pigment;
wherein the composition comprises at least 25% of a high coverage pigment selected from the group consisting of titanium dioxide, zinc oxide, and mixtures thereof,
wherein the amount of high coverage pigment includes the titanium dioxide coating of the titanium coated pigment; and
wherein when the high coverage pigment is used as the core pigment of the metal soap coated pigment, such amount of high coverage pigment is also included.

2. The composition of claim 1 wherein the titanium coating accounting for at least 40% of the titanium coated pigment.

3. The composition of claim 1 wherein the metal soap coating accounting for at least 3% of the metal soap coated pigment.

4. The composition of claim 1 wherein the powder comprises colored powders.

5. The composition of claim 4 which is designed to be used with a foundation, the foundation having a color, and wherein the CMC DE of the foundation color and the concealer color is less than 1.5.

6. The composition of claim 1 comprising by weight from about 75% to about 99% of powders, the powders comprising by weight of the entire composition:
(a) from about 1% to about 60% of the titanium coated pigment, the titanium coating accounting for at least 40% of the titanium coated pigment;
(b) from about 1% to about 70% of the metal soap coated pigment, the metal soap coating accounting for at least 3% of the metal soap coated pigment; and
by weight from about 1% to about 25% of a binder selected from the group consisting of non-volatile oil, lipophilic surfactant, humectant, and mixtures thereof;
wherein the composition comprises from about 25% to about 60% of the high coverage pigment.

7. A method of making up the skin comprising the steps of:
(1) applying to the skin a foundation; and
(2) applying to the skin the concealer composition of claim 1.
